(19) United States
(12) Patent Application Publication
(10) Pub. No.: US 2012/0005925 A1
(43) Pub. Date: Jan. 12, 2012

(54) Orthotic Devices and Methods of Manufacture

(76) Inventor: Monica Aho, Chassell, MI (US)

(21) Appl. No.: 12/961,354

(22) Filed: Dec. 6, 2010

Related U.S. Application Data
(60) Provisional application No. 61/399,415, filed on Jul. 12, 2010.

(51) Int. Cl. A61F 5/14

(52) U.S. Cl. 36/140

(57) ABSTRACT

The invention utilizes a semi-rigid endoskeleton foot support encased inside an elastomeric sock. The endoskeleton controls the hind foot as well as supporting the medial longitudinal arch, extending over the malleoli if extra support is needed for additional biomechanical control, while allowing the fore, lateral, and rear plantar surface of the foot indirect contact with the ground through the sock. The elastomeric sock provides circumferential compression, while simultaneously securing the endoskeleton intimately to the wearer's foot.

### PRIORITY CLAIM

This application claims the benefit of U.S. provisional patent application Ser. No. 61/399,415, filed Jul. 12, 2010, which is incorporated herein by reference.

### FIELD OF THE INVENTION

This invention relates generally to ankle-foot orthotics and corrective footwear, and more specifically to an orthotic sock for the correction of flexible pes planus and decreased proprioceptive awareness.

### BACKGROUND OF THE INVENTION

Ankle-Foot-Orthotics (AFOs) are commonly used to improve the biomechanical alignment of the lower extremity, enhance the efficiency of gait, and improve balance in children with disabilities and developmental delays. Current orthoses on the market have not yet been able to combine intimate support of the foot with improved proprioceptive feedback to address the deficits associated with flexible pes planus (flat foot), as is often seen in children with joint laxity or decreased muscle tone. Proprioception is the ability of a person to know where a body part is in space. Proprioceptors are those sensory receptors located in muscles, joints, and tendons that provide information about where a body part is in relation to the body as a whole, and whether or not the body part is moving. Excessive joint laxity results in a decreased stimulation of proprioceptors and, subsequently, a decreased awareness of joint position. Currently-available orthotics and supramalleolar orthotics fail to address the sensory needs of the foot as a means to improve its function. Orthotics on the market today are bulky, necessitating the purchase of shoes several sizes bigger than the wearer's foot to fit over the orthotics. Should an orthotic be needed for only one foot, two different sized shoes may need to be purchased, or a lift placed into the opposite shoe to prevent a difference in apparent leg length. Furthermore, currently available orthotics must be worn with shoes in order to derive benefit. Many orthotics are made of thermoplastic, which is inherently hot and non-breathable. An additional drawback to such orthotics is the risk of pressure sores over bony prominences, and a looser fit, which causes gapping. Often, traditional designs are rigid, and do not allow a significant degree of foot accommodations to occur to the supporting surface, which leads to muscle wasting and atrophy in the intrinsic and extrinsic foot and calf musculature. Current orthotics create a barrier between the wearer's foot and the ground, because they run along the entire plantar surface of the foot, which lessens the sensory feedback the wearer receives from the supporting surface. Lastly, due to bulky, obvious designs, many people refuse to wear the braces because of the stigma of disability.

### BRIEF DESCRIPTION OF THE DRAWINGS

FIG. 1a illustrates a Medial view of Orthotic Version Endoskeleton according to one embodiment.

FIG. 1b illustrates a Plantar view of Orthotic Version Endoskeleton according to one embodiment.

FIG. 1c illustrates a Lateral view of Orthotic Version Endoskeleton according to one embodiment.

FIG. 2a illustrates a Medial view of Supramalleolar Version Endoskeleton according to one embodiment.

FIG. 2b illustrates a Plantar view of Supramalleolar Version Endoskeleton according to one embodiment.

FIG. 2c illustrates a Posterior view of Supramalleolar Version Endoskeleton according to one embodiment.

FIG. 2d illustrates a Lateral view of Supramalleolar Version Endoskeleton according to one embodiment.

FIG. 3a illustrates a Medial Exterior view of Orthotic Version according to one embodiment.

FIG. 3b illustrates a Bottom Exterior view of Orthotic Version according to one embodiment.

### DETAILED DESCRIPTION OF THE PREFERRED EMBODIMENT

An orthotic encased inside an elastomeric sock for the purpose of controlling the foot and ankle of a person with flexible pes planus (flat foot) and/or poor proprioceptive feedback. The invention is comprised of two components. The first component is a semi-rigid support, herein referred to as an endoskeleton, because it provides an internal structure in relation to an external sock. The support could also be construed as an “exoskeleton” in relation to the user's foot, as it follows the longitudinal medial arch of the foot, wrapping around the posterior calcaneus and ending on the lateral aspect of the foot. Should additional support be required by the wearer, the endoskeleton may extend proximally over the malleoli, with cutouts around the bony prominences of the malleoli, to prevent pressure areas. The lateral, fore, and rear plantar aspects of the foot are left uncovered by the endoskeleton. An elastomeric sock surrounds the endoskeleton, encases the entire foot with circumferential compression, and proceeds proximally up the ankle and leg to secure the endoskeleton to the wearer's foot. The compressive sock may include a flap along the instep which can be opened to make the sock looser, thereby easier to don and doff. The flap can alternatively be pulled taut and secured along the lateral border of the sock to customize the amount of circumferential compression along the medial longitudinal arch.

The orthotic easily fits into a traditional shoe, but doesn't have to be worn with shoes in order to incur benefit. The present invention is slim enough that shoes do not have to be purchased in a significantly bigger size to accommodate the orthotic. Because the endoskeleton will not encroach upon the weight-bearing surface of the foot, it will not add height to the user's leg, and will not create an apparent leg length discrepancy should only one foot require use of the orthotic. The possibility of pressure points is reduced by providing structural support only where absolutely necessary for biomechanical correction, avoiding contact with bony prominences. This allows for increased comfort and decreased secondary complications arising from potential pressure sores. Furthermore, the orthotic is breathable and lightweight, as the majority of the user's foot is covered only by a porous sock and not an occlusive, non-porous orthotic. The orthotic provides circumferential compression that stimulates joint proprioceptors, for improved sensory feedback. The orthotic also allows for normalized foot biomechanics by facilitating congruency of the tarsal and metarsal bones of the user's foot, for improved lower extremity function. The invention improves kinesthetic awareness of the ground by allowing the weight-bearing surface of the user's foot indirect contact through the sock, for improved postural stability. The orthotic encourages intrinsic and extrinsic foot and ankle muscle strengthening by allowing normalized movement of the foot, rather than blocking out movement patterns. Also, the orthotic resembles a typical pair of socks, thereby increasing compliance of use with the wearer by avoiding a disabled stigma.

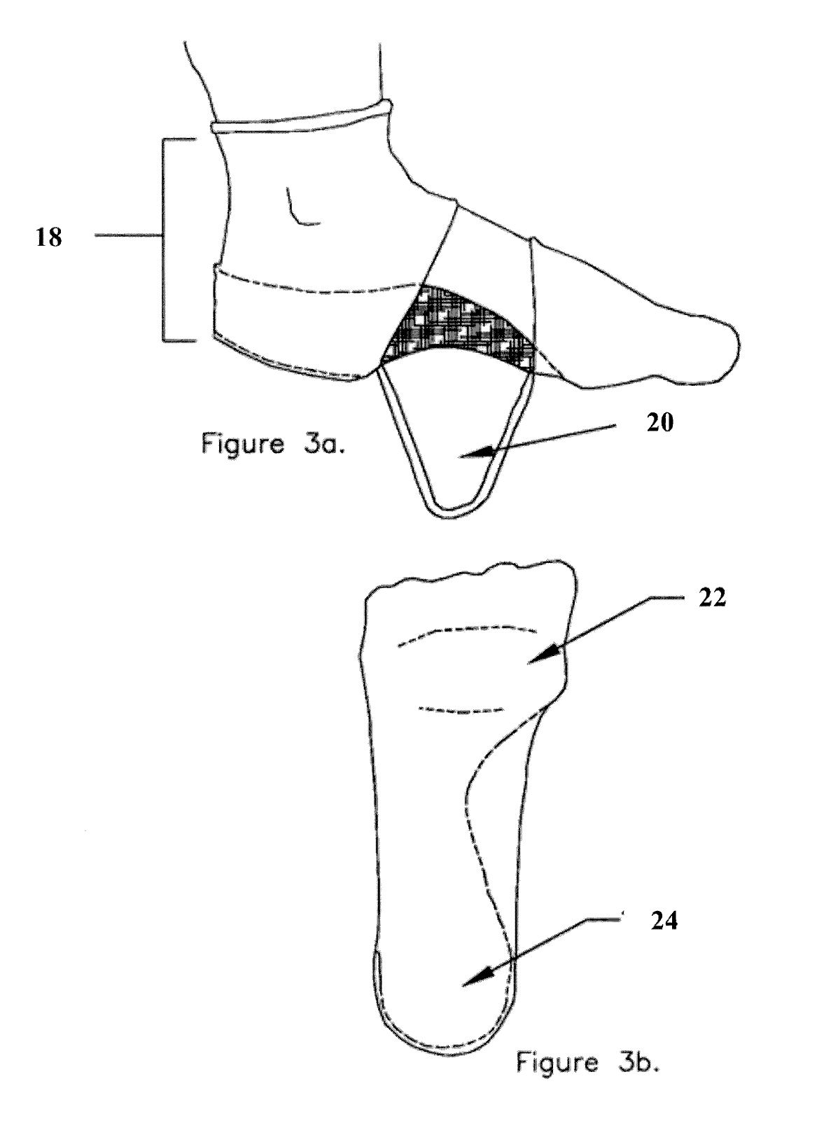

The present invention is a foot orthotic best understood upon viewing the preferred embodiments illustrated in the Figures. In FIGS. 3a and 3b, an elastomeric sock encases a semi-rigid endoskeleton 4. The elastomeric sock 18 may comprise Latex®-free, breathable, and washable materials that provide enough compression to the wearer's foot to approximate joint surfaces, provide a small amount of give in response to movement, and return to their original shape after deformation. An example would be a fabric providing a minimum of 20 mmHg of compression, and comprised of silicone, nylon, and/or lycra blends. Compression stimulates joint proprioceptors, providing the user with increased sensory feedback from the foot. Compression additionally approximates the joint surfaces of the tarsal and metatarsal bones of the foot, making the joints of said bones more congruent, thus allowing the mechanism of the foot as a whole to act more efficiently.

In one embodiment, a flap 20 of similar or other material may be sewn to the medial aspect of the sock, to be pulled taut and secured to the lateral aspect of the sock by the wearer via a method such as a hook and loop enclosure. This flap is configured to provide customized circumferential compression when secured, and allow for ease of donning and doffing by loosening compression of the sock when unsecured. The heel 24 of said elastomeric sock may be reinforced by an additional piece of fabric for improved durability, as this is often an area that wears through with prolonged use.

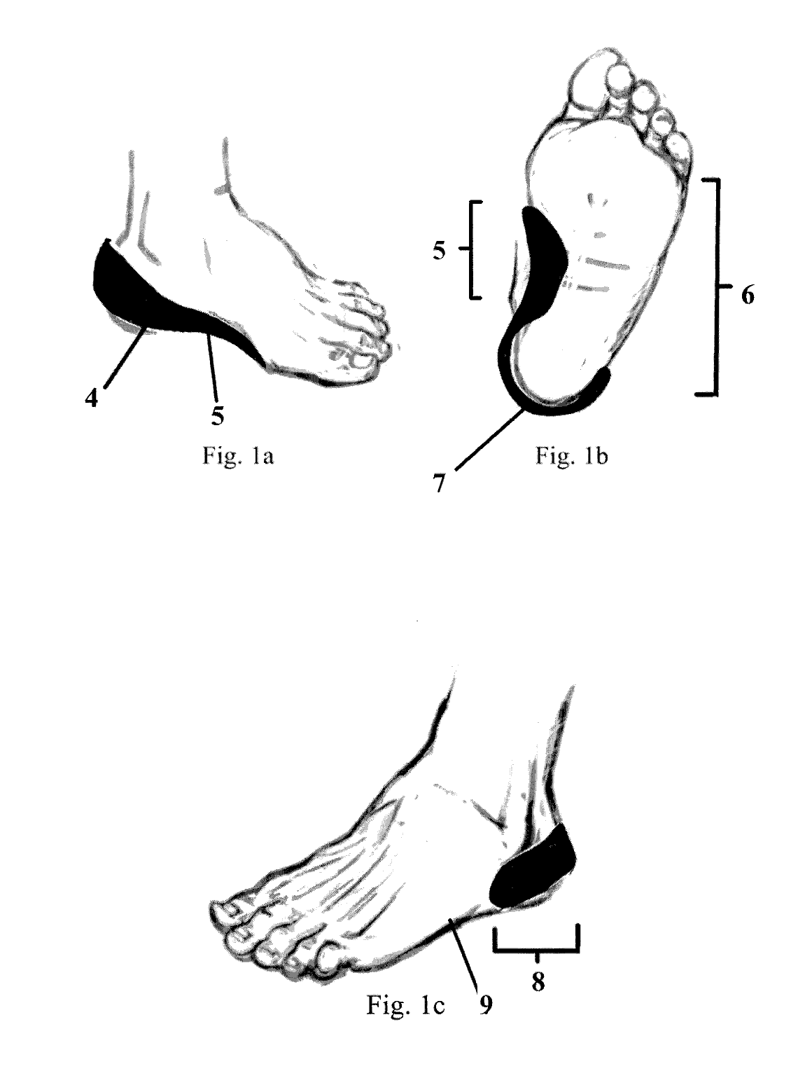

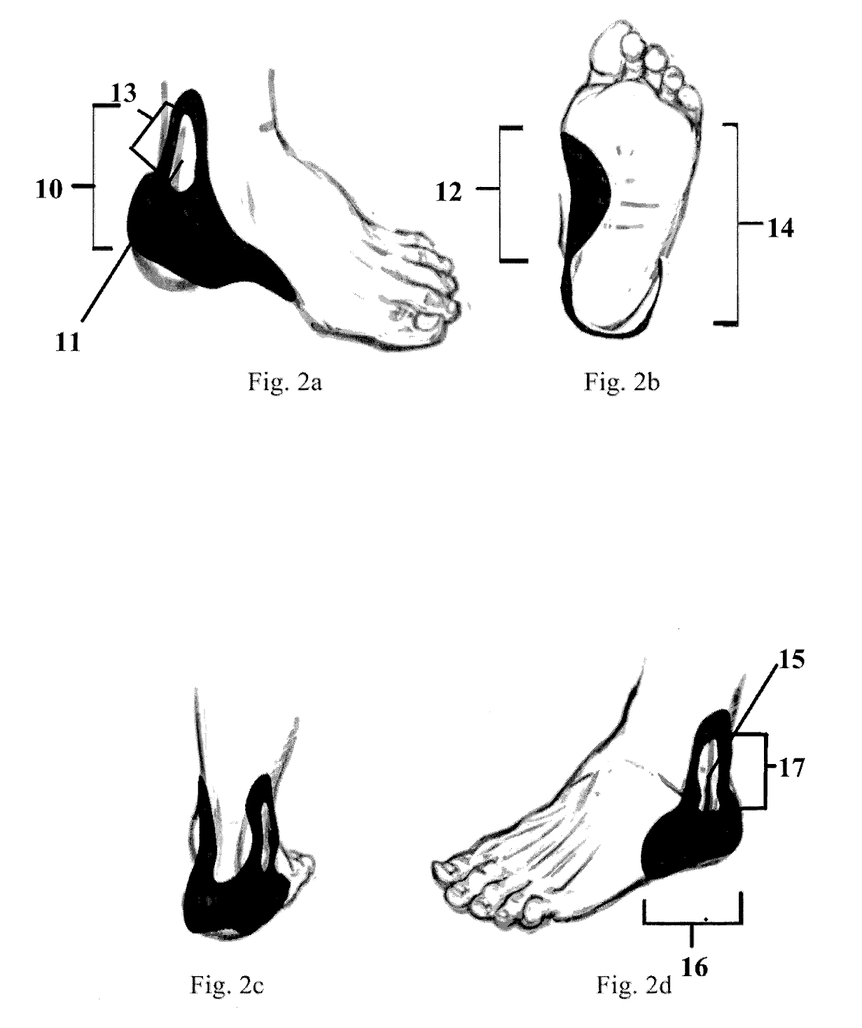

The sock encases a semi-rigid endoskeletal support system 4 (see FIGS. 1a, 1b, and 1c), made of a material such as carbon fiber hardened with polyurethane resin. Using ASTM D2240 classifications of hardness, a Shore D Durometer 80 resin provides enough hardness to counteract the biomechanical forces of a weighted foot collapsing into hyper-pronation. Carbon is thin and lightweight, yet extremely strong, and can provide the necessary support without becoming cumbersome. The skeletal framework supports the foot along the medial longitudinal arch 5, controls the hind foot from the posterior (rather than plantar) aspect by wrapping around the posterior calcaneus 7 and ending on the lateral aspect 8 of the wearer's foot (FIGS. 1a, 1b, and 1c). When the user is not wearing shoes, this configuration allows the fore, lateral, and rear plantar surfaces 6 of the foot to remain indirectly in contact with the ground through the sock 22 for the purpose of improved kinesthestic feedback (FIG. 1b), approximating the conditions of a foot wearing a typical sock. The endoskeleton may be lined on the inside with thin, dense foam to improve the wearer's comfort when utilizing the device. Should the wearer require more biomechanical control, a second embodiment raises the medial and lateral aspects 10 and 16 of the endoskeleton 4 proximal to the malleoli 11 and 15 (FIGS. 2a, 2b, 2c, and 2d). Cutouts 13 and 17 around the malleoli prevent pressure areas from developing. The remainder of the endoskeleton may be the same as in FIGS. 1a, 1b, and 1c.

Thus the reader can see how the invention remediates many of the common problems associated with currently available orthotics. The described orthotic constitutes a breathable, light-weight, non-disabled appearing foot support system that provides adequate biomechanical support only where needed to improve alignment. By blocking only hyperpronation, normal degrees of pronation, supination, and extension of the toes are allowed, which leads to a strengthening rather than weakening of extrinsic and intrinsic foot musculature. The invention's slim profile allows for the user to wear their typical shoes, yet the orthotic does not have to be worn with shoes for the user to derive benefit. The invention improves the sensory feedback of the wearer through compression of joint surfaces to stimulate the proprioceptors of the foot, as well as allows the wearer increased kinesthetic feedback from the supporting surface. Because the invention's endoskeleton avoids contact with many of the bony prominences of the foot, the probability of pressure sores is greatly decreased.

The invention is a semi-custom orthotic, and may be constructed using a positive mold of the user's foot. The process of positive mold construction is of known convention to those skilled in the art, wherein a cast is made of the user's foot while in a talar-neutral position, and a Plaster-of-Paris positive model is created from the cast. Two to three layers of resin-soaked carbon fiber are formed over the positive mold in the desired pattern (see FIGS. 1a, 1b, 1c, 2a, 2b, 2c, and 2d), with each layers' fibers oriented 45 degrees to the previous to provide multi-directional strength to the endoskeleton. Once cured and hardened, the endoskeleton is fitted to the patient by grinding it down for comfort and appropriate support. The endoskeleton is then lined with a thin layer of dense foam for comfort, with edges tapered to prevent pressure areas. A compression sock commensurate to the user's foot is modified with an open area over the proximal dorsum of the foot, and a flap along the instep to allow for ease of donning and doffing, as well as to customize circumferential compression. The flap can thus be manually pulled over the dorsum of the foot, and secured to the lateral side of the sock by the wearer, using a method such as a hook and look attachment. The endoskeleton is secured inside the sock in one embodiment, again by a method such as a hook and loop attachment, to prevent slippage, and allow separation of the endoskeleton from the sock for laundering

While the above description contains many specificities, these should not be construed as limitations on the scope of the invention, but rather as an exemplification of one preferred embodiment. Many other variations are possible. The endoskeleton can be made of other materials that provide structural support with dynamic capabilities. The sock fabric itself can be made from other fabrics that provide the necessary compression, comfort, breathability, and elasticity. A further embodiment might achieve the appropriate compression without the need for a flap, or utilize other configurations to secure the endoskeleton to the user's foot. Such configurations could include all or a portion of a compressive sock, a bootie, a strapping system, an elastic band, or any other compressive device that stimulates joint proprioceptors while securing the orthotic to the user's foot. Another embodiment could extend the endoskeleton's support even more proximally up the user's leg to impact control of the knee joint. It is believed that the present invention will probably be most beneficial for pediatric clients with excessive joint laxity, given their lower body mass, and the likelihood that their feet are more pliable and have less rigid deformities; however, further developments may support the use of the invention with older clients, as well as those demonstrating a wide variety of symptoms (i.e. ataxia, choreiform movements, poor proprioceptive awareness due to sensory processing dysfunctions, flaccidity or weakness due to spinal cord injury or spina bifida, to name a few). Accordingly, the scope of the invention should be determined not by the embodiments illustrated, but by the claims and their legal equivalents.

### DRAWINGS/REFERENCE NUMBERS

4—Orthotic endoskeleton providing semi-rigid support to medial longitudinal arch and medial calcaneus
5—The medial, longitudinal arch
6—The fore, lateral, and rear foot are left unencumbered by the endoskeleton to allow contact with the ground via sock or shoe
8—Lateral calcaneal support ends proximal to the base of the fifth metatarsal
9—Base of the fifth metatarsal
10—Endoskeleton raised proximal to the medial malleolus for increased medial control
11—Medial Malleolus
12—Support along the medial longitudinal arch and medial calcaneus
13—Cutout around the medial malleolus
14—The fore, lateral, and rear foot unencumbered by the endoskeleton to allow contact with the ground indirectly through the sock
15—Lateral malleolus
16—Endoskeleton raised above the lateral malleolus for increased lateral control
17—Cutout around the lateral malleolus
18—Elastomeric sock securing endoskeleton to foot and providing circumferential compression
20—Flap with hook and loop edges
22—Elastomeric sock is the only thing between the weight-bearing surface of the wearer's foot and the ground
24—Reinforced heel of sock for durability

I claim:

1. An orthotic foot support comprising:
an elastomeric sock; and
a semi-rigid endoskeleton encased inside said sock; and
wherein the endoskeleton is configured to extend along the medial longitudinal arch of a user's foot when the orthotic foot support is worn by the user.

2. The orthotic foot support of claim 1 wherein the orthotic foot support is configured so that the elastomeric sock is in direct physical contact with the fore, lateral, and rear plantar aspects of the user's foot when the orthotic foot support is worn by the user.

3. The orthotic foot support of claim 1 wherein the endoskeleton is configured to wrap around the posterior calcaneus of the user's foot when the orthotic foot support is worn by the user.

4. The orthotic foot support of claim 1 wherein the endoskeleton is configured so as to extend along a lateral aspect of the user's foot when the orthotic foot support is worn by the user.

5. The orthotic foot support of claim 1 wherein the elastomeric sock is configured to force the endoskeleton against the user's foot when the orthotic foot support is worn by the user.

6. The orthotic foot support of claim 1 wherein the elastomeric sock is configured so as to substantially and closely conform to the user's foot when the orthotic foot support is worn by the user, and is configured to provide compression to joint structures of the user's foot.

7. The orthotic foot support of claim 1 wherein the elastomeric sock comprises a fabric configured to stretch to a slight degree against resistance and then return to its original shape once the resistance is removed.

8. The orthotic foot support of claim 1 wherein a flap is permanently attached to the medial portion of the elastomeric sock and is configured so as to allow it to be pulled across the dorsum of the user's foot to releasably attach to the lateral portion of the elastomeric sock, so as to customize the amount of circumferential compression around the instep of the user's foot.

9. The orthotic foot support of claim 1 wherein the endoskeleton is configured to extend up the user's leg proximal to the user's medial and lateral malleoli.

10. The orthotic foot support of claim 9 wherein the endoskeleton does not contact the bony prominences of the malleoli of the user when the orthotic foot support is worn by the user.

11. An orthotic foot support comprising a semi-rigid structure configured to be worn with an elastomeric sock; wherein the structure extends along the medial longitudinal arch of a user's foot when the orthotic foot support is worn by the user.

12. The orthotic foot support of claim 11 wherein the structure is configured such that the fore, lateral, and rear plantar aspects of a user's foot are in contact with the supporting surface when the orthotic foot support is worn by the user.

13. The orthotic foot support of claim 11 wherein the structure is configured to wrap around the posterior calcaneus of the user's foot when the orthotic foot support is worn by the user.

14. The orthotic foot support of claim 11 wherein the structure is configured so as to extend along a lateral aspect of the user's foot when the orthotic foot support is worn by the user.

15. An orthotic foot support comprising:
a semi-rigid endoskeleton; and
a compressive device configured to secure said endoskeleton to a user's foot; wherein the endoskeleton is configured to extend along the medial longitudinal arch of the user's foot when the orthotic foot support is worn by the user.

16. The orthotic foot support of claim 15 wherein the endoskeleton is configured so that the fore, lateral, and rear plantar aspects of the user's foot are left uncovered by the endoskeleton when the orthotic foot support is worn by the user.

17. The orthotic foot support of claim 15 wherein the endoskeleton is configured to wrap around the posterior calcaneus of the user's foot when the orthotic foot support is worn by the user.

18. The orthotic foot support of claim 15 wherein the endoskeleton is configured so as to extend along a lateral aspect of the user's foot when the orthotic foot support is worn by the user.

19. The orthotic foot support of claim 15 wherein the compressive device is configured to force the endoskeleton against the user's foot when the orthotic foot support is worn by the user.

20. The orthotic foot support of claim 15 wherein the compressive device is configured so as to substantially and closely conform to all or a portion of the user's foot when the orthotic foot support is worn by the user, and is configured to provide compression to joint structures of the user's foot.
